(19) United States
(12) Patent Application Publication
(10) Pub. No.: US 2025/0281072 A1
(43) Pub. Date: Sep. 11, 2025

(54) INSERTION DEVICE FOR A BIOSENSOR

(71) Applicant: BIONIME CORPORATION, Taichung City (TW)

(72) Inventors: Chun-Mu HUANG, Taichung City (TW); Guan-Reui Li, Taichung City (TW); Chien-Chih Liao, Taichung City (TW); Ting-Yu Liu, Taichung City (TW)

(73) Assignee: BIONIME CORPORATION, Taichung City (TW)

(21) Appl. No.: 19/069,563

(22) Filed: Mar. 4, 2025

Related U.S. Application Data
(60) Provisional application No. 63/561,355, filed on Mar. 5, 2024.

(51) Int. Cl. A61B 5/145; A61B 5/00

(52) U.S. Cl. CPC A61B 5/14503; A61B 5/14532; A61B 5/6849

(57) ABSTRACT

An insertion device includes a cover assembly, an implant module, a patch base, a sensor assembly, and a peel-off component. The cover assembly includes an outer shell and a bottom cover. The outer shell defines an accommodating space. The implant module is disposed in the accommodating space and is operable for implanting and extracting a needle. The patch base includes a base body, an adhesive pad that is connected fixedly to the base body, and an outer release layer that is attached detachably to the adhesive pad. The sensor assembly is connected to the base body when the needle is implanted by the implant module. The peel-off component is connected to the bottom cover and the outer release layer. The peel-off component removes the outer release layer from the adhesive pad when the bottom cover is separated from the outer shell.

### CROSS-REFERENCE TO RELATED APPLICATION

This application claims the benefit of U.S. Provisional Patent Application No. 63/561,355, filed on Mar. 5, 2024, the entire disclosure of which is incorporated by reference herein.

### FIELD

The disclosure relates to biological detection system and method that are applied to an organism, and more particularly to an insertion device for installing a biological detection device onto the skin of the organism and for implanting a biosensor under the skin of the organism.

### BACKGROUND

Conventional self-testing of blood glucose is conducted through drawing blood from capillaries of the fingertip, disposing the blood on a blood glucose test strip, and using a machine to read the blood glucose level. As long as the test is conducted correctly, the blood glucose level obtained will be very accurate. When the obtained blood glucose level is too high or too low, medical care can be given as soon as possible. Some people may need to test their blood glucose regularly, especially patients who have poor blood glucose control, are under insulin treatment, or have large blood glucose fluctuations.

Another device can also achieve self-testing of blood glucose, which is a continuous glucose monitor (CGM). A sensor needle is disposed underneath the patient's skin to continuously monitor glucose concentration in tissue fluid (glucose in blood diffuses into tissue fluid to enter cells). The sensor estimates blood glucose levels via calculation, and can provide immediate blood sugar level values. The sensor periodically records blood glucose levels, and shows a trend chart of the changes in blood glucose, so as to send out a warning when the blood glucose level is too high or too low.

Current studies show that, for patients who, with type 1 or type 2 diabetes, need insulin injections, using the CGM for testing, in comparison to testing blood glucose through drawing blood from the fingertip, lowers glycohemoglobin by 0.6% and decreases a total amount of time during which low blood glucose occurs each day.

Since the CGM is worn by a user for long periods of time, the trend of reducing the size of the CGM is inevitable. A structure of the CGM includes: (a) a sensor for detecting a biosignal that corresponds to glucose concentration in the human body, (b) a receiver for receiving and transmitting the biosignal, and (c) an insertion device for attaching the sensor to the receiver, and for inserting the sensor under the user's skin.

To achieve safe and accurate implantation of the sensor under the user's skin, and to have the biosignal sensed by the sensor transmitted to corresponding receiving equipment of the receiver to allow the user to obtain the state of their blood glucose at any time, the applicant has filed two applications, U.S. Patent Application Publications No. 20210030960A1 and No. 20210030344A1. However, the applicant believes that the functions of the insertion devices disclosed in the abovementioned applications can be further improved to provide more convenient use and a shorter period of sensor implementation time. The applicant hopes the improved insertion device can make assembly easier in manufacturing and production processes to increase production yield and speed.

### SUMMARY

Therefore, an object of the disclosure is to provide an insertion device that can alleviate at least one of the drawbacks of the prior art.

According to the disclosure, the insertion device is adapted to be installed in a biosensor. The insertion device includes a cover assembly, an implant module, a patch base, a sensor assembly, and a peel-off component. The cover assembly includes an outer shell and a bottom cover that is removably coupled to the outer shell. The outer shell defines an accommodating space. The implant module is disposed in the accommodating space and is operable for implanting and extracting a needle. The patch base is positioned removably relative to the implant module, and includes a base body, an adhesive pad, and an outer release layer. The adhesive pad is connected fixedly to the base body. The outer release layer is attached detachably to the adhesive pad. The sensor assembly is connected to the base body when the needle is implanted by the implant module. The peel-off component is connected to the bottom cover and the outer release layer. The peel-off component removes the outer release layer from the adhesive pad when the bottom cover is separated from the outer shell.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a sectional perspective view illustrating an embodiment of an insertion device according to the disclosure.

FIG. 2 is a sectional perspective view taken along line II-II in FIG. 1.

FIG. 3 is a sectional view taken along line III-III in FIG. 1, illustrating a configuration of a plurality of positioner columns.

FIG. 4 is a partially exploded perspective view of the embodiment.

FIG. 5 is a fragmentary exploded perspective view of the embodiment.

FIG. 6 is a perspective view of an inner lining member of the embodiment.

FIG. 7 is a perspective view of a main body member of the embodiment.

FIG. 8 is an exploded schematic perspective view of an insertion seat, a needle extraction member, and the positioner columns of the embodiment.

FIG. 9 is a fragmentary sectional view of the insertion seat.

FIG. 10 is a top view of the needle extraction member.

FIG. 11 is a bottom view of a peel-off component and a patch base of the embodiment.

FIG. 12 is an exploded perspective view of the peel-off component and the patch base.

FIG. 13 is a sectional view of the embodiment in an unfired state, illustrating a bottom cover of the embodiment being attached to an outer shell of the embodiment.

FIG. 14 is a sectional view taken along line XIV-XIV in FIG. 13.

FIG. 15 is a schematic sectional view of the embodiment in a pre-firing state, illustrating the bottom cover being separated from the outer shell.

FIG. 16 is a schematic sectional view, illustrating the outer shell being pressed after the bottom cover has been removed, and the embodiment being in a firing state.

FIG. 17 is a sectional view taken along line XVII-XVII in FIG. 16.

FIG. 18 is a schematic sectional view of the embodiment in the firing state, illustrating a firing restriction structure being unlocked.

FIG. 19 is a sectional view taken along line XIX-XIX in FIG. 18.

FIG. 20 is a fragmentary schematic sectional view of the embodiment, illustrating a needle being fired.

FIG. 21 is a sectional view taken along line XXI-XXI in FIG. 20.

FIG. 22 is a fragmentary schematic sectional view of the embodiment in a post-fired state.

FIG. 23 is a sectional view taken along line XXIII-XXIII in FIG. 22.

FIG. 24 is a fragmentary schematic sectional view of the embodiment, illustrating the needle being extracted.

FIG. 25 is a sectional view taken along line XXV-XXV in FIG. 24.

FIG. 26 is a fragmentary schematic sectional view of the embodiment after the patch base, a sensor assembly, and the main body member have been removed.

### DETAILED DESCRIPTION

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

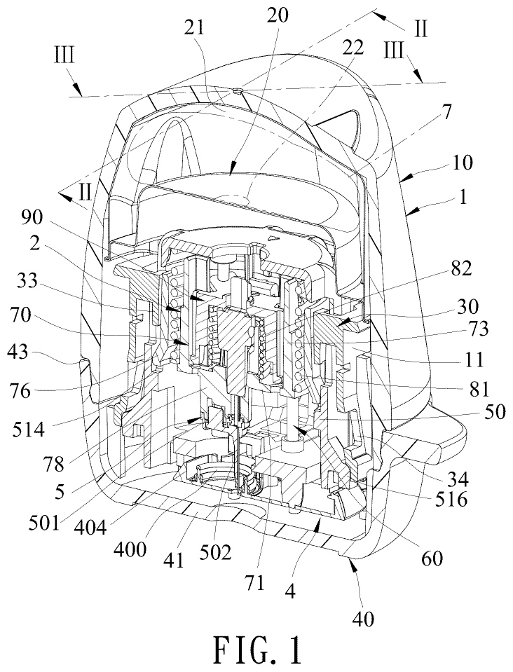

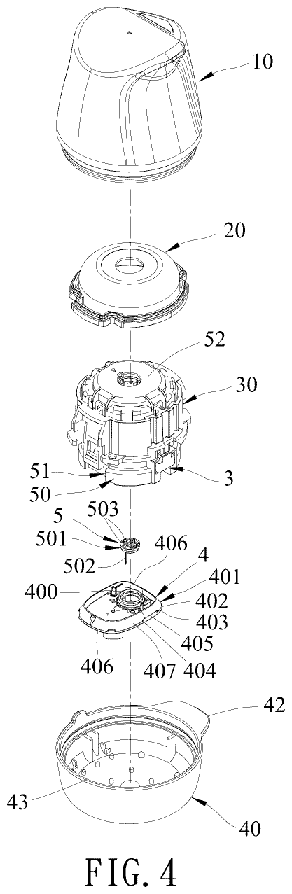

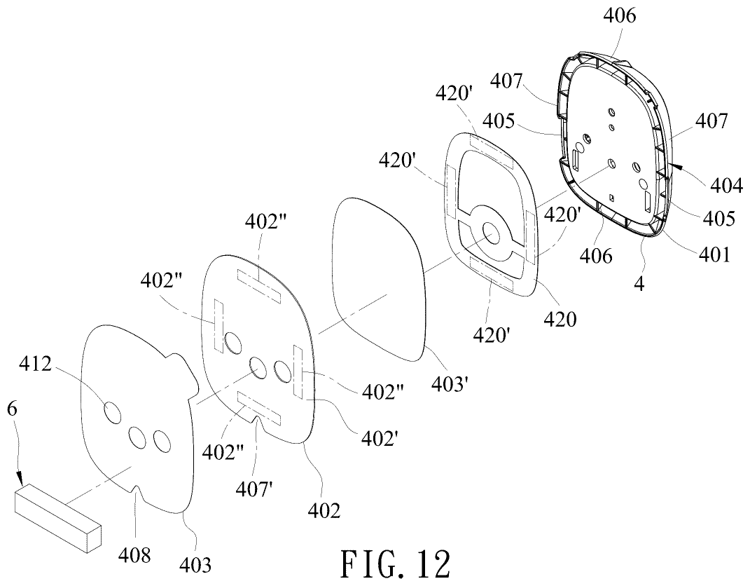

Referring to FIGS. 1 to 5, an embodiment of an insertion device for a biosensor according to the disclosure includes a cover assembly 1, an implant module 2, two fixture members 3, a patch base 4, a sensor assembly 5, a peel-off component 6 (see FIG. 12), and a desiccant 7.

The cover assembly 1 includes an outer shell 10, a top cap 20 fixed in the outer shell 10, an inner lining member 30 disposed in the outer shell 10 and at a side of the top cap 20, and a bottom cover 40 airtightly and removably coupled to the outer shell 10. The outer shell 10 defines an accommodating space 11. The top cap 20 and the outer shell 10 cooperatively define a receiving space 21 that takes up a portion of the accommodating space 11, and that is isolated from the remaining portions of the accommodating space 11. The top cap 20 has an open hole 22 for disposing the desiccant 7 (which may be, for example, a desiccant pack, a desiccant tablet, or desiccant beads) into the receiving space 21. In this embodiment, the desiccant 7 is smaller than the open hole 22. During assembly of the insertion device, after the desiccant 7 is disposed in the receiving space 21, the open hole 22 is blocked (e.g., by an additional component) but not sealed, so the desiccant 7 is still able to keep the contents in the insertion device dry. In other embodiments, if the desiccant 7 is a desiccant package that is bigger than the open hole 22, there will be no need to block the open hole 22 by an additional component.

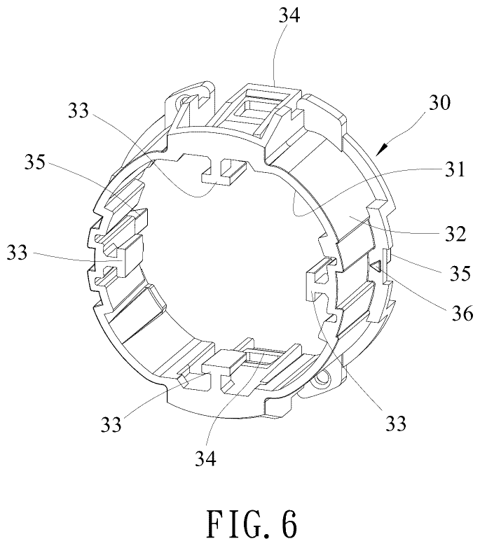

Referring to FIG. 6, the inner lining member 30 is hollow and ring-shaped, and has an inner peripheral surface 31, an outer peripheral surface 32 opposite to the inner peripheral surface 31, a plurality of firing portions 33 disposed on the inner peripheral surface 31, a pair of latch portions 34 being elastic and extending downwards from the outer peripheral surface 32, a pair of push members 35 disposed at a bottom end of the inner lining member 30 and extending downwardly, and a fourth alignment label 36 disposed on the outer peripheral surface 32. The fourth alignment label 36 is a triangular label.

The bottom cover 40, when being coupled to the outer shell 10, faces an opening of the accommodating space 11. The bottom cover 40 includes a bottom plate portion 41.

The implant module 2 is disposed in the accommodating space 11 of the cover assembly 1. The implant module 2 includes a main body unit 50, a positioner unit 60, an implant seat 70, a first resilient member 81, a needle extraction member 90, a second resilient member 82 and a needle implant member 100.

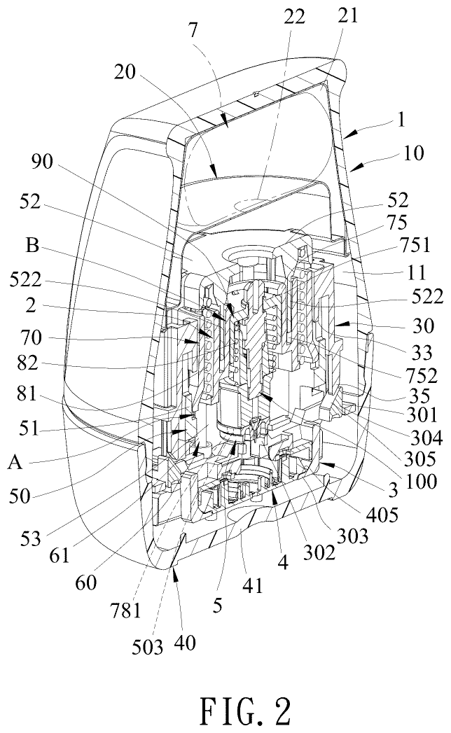

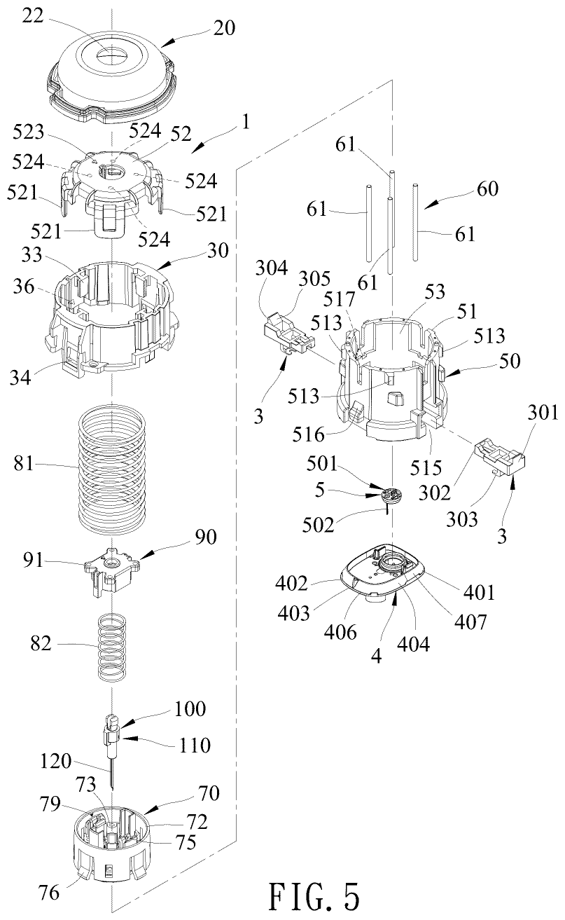

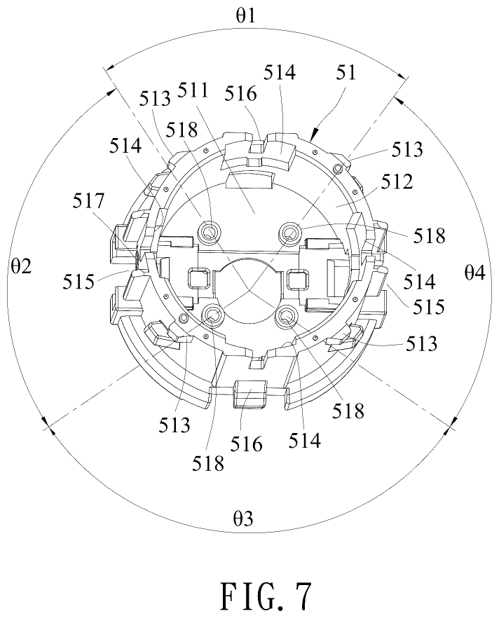

The main body unit 50 is connected to the cover assembly 1, and is slidably disposed in the inner lining member 30. The main body unit 50 includes a main body member 51, and a main body cap 52 that is removably connected to the main body member 51. The main body member 51 and the main body cap 52 cooperatively define a movement space 53. Referring to FIG. 7, the main body member 51 has a bottom wall 511, an annular wall 512 connected to the bottom wall 511, a plurality of engagement portions 513 disposed on the annular wall 512, a plurality of buckle stopper portions 514 formed on the annular wall 512, a pair of slide grooves 515 extending through the annular wall 512, recessing into the bottom wall 511, and spatially communicating with the movement space 53, a pair of lock portions 516 extending from the annular wall 512 and respectively corresponding in position to the latch portions 34, and a first alignment label 517 disposed on the annular wall 512. The bottom wall 511 has four lower positioning holes 518. An angle is formed between every two adjacent lower positioning holes 518, and there are four angles (θ₁, θ₂, θ₃, θ₄). The angles (θ₁, θ₂, θ₃, θ₄) are not symmetrically arranged. The first alignment label 517 corresponds in position to the fourth alignment label 36. The first alignment label 517 is a triangular label. Referring to FIG. 5, the main body cap 52 has a plurality of connection portions 521 that are respectively engaged with the engagement portions 513, two limiter members 522 (see FIG. 2) that are elongated, a second alignment label 523 that corresponds in position to the first alignment label 517, and four upper positioning holes 524. The second alignment label 523 is a triangular label. An angle is formed between every two adjacent upper positioning holes 524, and there are four angles. The angles formed between every two adjacent upper positioning holes 524 are respectively identical to the angles (θ₁, θ₂, θ₃, θ₄) formed between every two adjacent lower positioning holes 518, and are not symmetrically arranged.

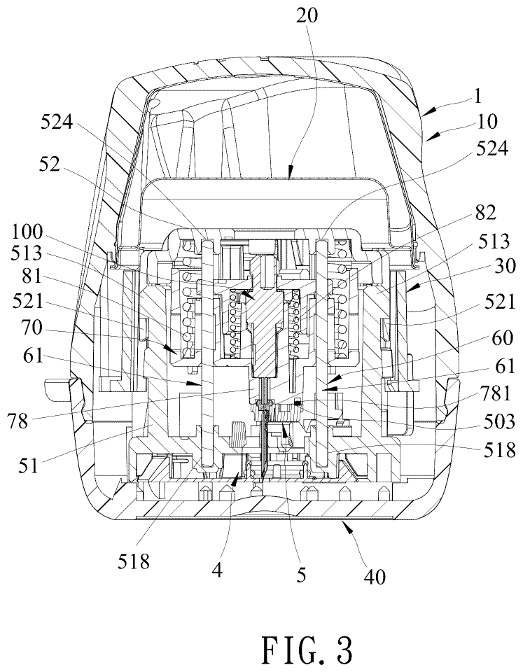

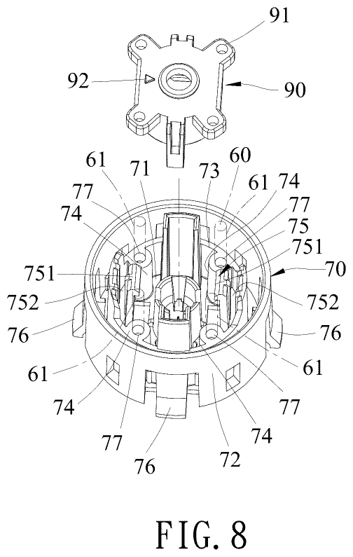

Referring to FIG. 8, the positioner unit 60 is disposed in the movement space 53 (see FIG. 5), and includes four positioner columns 61 that interconnect the main body member 51 (see FIG. 5) and the main body cap 52 (see FIG. 5). The positioner columns 61 extend in an axial direction. Referring to FIG. 3, each of the positioner columns 61 has an end disposed in a respective one of the lower positioning holes 518, and an axially-opposite end disposed in a respective one of the upper positioning holes 524. An angle is formed between every two adjacent positioner columns 61, and there are four angles. The angles formed between every two adjacent positioner columns 61 are respectively identical to the angles (θ₁, θ₂, θ₃, θ₄) formed between every two lower positioning holes 518, and are not symmetrically arranged. That is to say, referring to FIG. 7, any one of the angles (θ₁, θ₂, θ₃, θ₄) is not equal to an opposite one of the angles (θ₃, θ₄, θ₁, θ₂). The positioner columns 61 are cylinders, but may be changed to other columns with different cross-section shapes as needed in other embodiments.

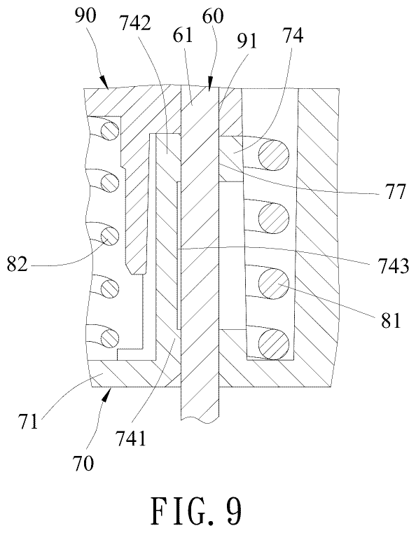

Referring to FIGS. 5 and 8, the implant seat 70 is removably disposed in the main body unit 50. The positioner columns 61 extend through the implant seat 70 so that the implant seat 70 is movable along the positioner columns 61 in the movement space 53. The implant seat 70 incudes a flat plate member 71, an outer barrel member 72 connected transversely to the flat plate member 71, an inner barrel member 73 connected transversely to the flat plate member 71 and disposed in the outer barrel member 72, a plurality of guide tubes 74 connected to the inner barrel member 73, a limiter module 75 connected to the inner barrel member 73, and positioning the needle extraction member 90 relative to the implant seat 70, a plurality of buckle portions 76 respectively engaged with the buckle stopper portions 514 of the main body member 51 and respectively driven by the firing portions 33 to be disengaged from the buckle stopper portions 514, four first guide holes 77, a positioner member 78 extending downwardly from the flat plate member 71 (see FIG. 3), and a fifth alignment label 79 (see FIG. 5) disposed on the outer barrel member 72 and corresponding in position to the first alignment label 517. The first guide holes 77 respectively extend through the guide tubes 74. The positioner columns 61 extend respectively through the four first guide holes 77. An angle is formed between every two adjacent first guide holes 77, and there are four angles. The angles formed between every two adjacent first guide holes 77 are respectively identical to the angles (θ₁, θ₂, θ₃, θ₄) formed between every two adjacent lower positioning holes 518, and are not symmetrically arranged (see FIG. 8, see FIG. 7 for the angles (θ₁, θ₂, θ₃, θ₄). Referring further to FIG. 9, each of the guide tubes 74 has a bottom portion 741 connected to the flat plate member 71, a top portion 742 opposite to the bottom portion 741 in the axial direction in which the positioner columns 61 extend, and a hollow portion 743 disposed between the bottom portion 741 and the top portion 742, and spatially communicating with a respective one of the first guide holes 77. The limiter module 75 includes a pair of limiter components 751 that are hooked in shape. Each of the limiter components 751 and the inner barrel member 73 cooperatively define a limiter slot 752. The limiter members 522 are respectively slotted in the limiter slots 752. The limiter module 75 restricts a position of the needle extraction member 90, so that the implant seat 70, the main body cap 52 and the needle extraction member 90 form a needle extraction limit structure (B). The buckle portions 76 are connected to the outer barrel member 72. The buckle portions 76 cooperate with the buckle stopper portions 514; that is to say, the buckle portions 76 engage the buckle stopper portions 514, so that the implant seat 70 and the main body member 51 form a needle implantation limit structure (A). The positioner member 78 has two link portions 781 (only one positioner member 78 is shown in FIG. 3) that are columns.

The first resilient member 81 has an end that abuts against the main body cap 52, and another end that abuts against the implant seat 70. The first resilient member 81 is a compression spring.

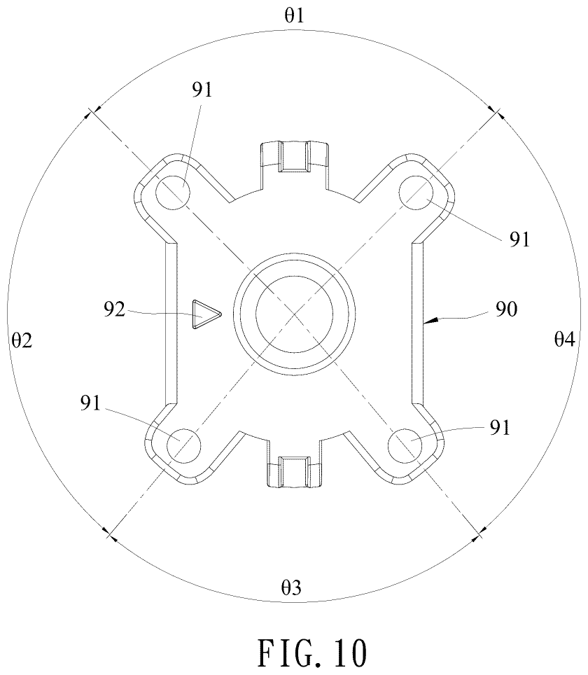

The needle extraction member 90 is separable from and positioned relative to the implant seat 70, and movement thereof is guided by the positioner unit 60. Specifically, the needle extraction member 90 has four second guide holes 91. The positioner columns 61 respectively extend through the second guide holes 91, so that the needle extraction seat 90 is slidable along the positioner columns 61. The needle extraction member 90 includes a third alignment label 92 that corresponds in position to the first alignment label 517. An angle is formed between every two adjacent second guide holes 91, and there are four angles. The angles formed between every two adjacent second guide holes 91 are respectively identical to the angles (θ₁, θ₂, θ₃, θ₄) formed between every two adjacent lower positioning holes 518, and are not symmetrically arranged (see FIG. 10).

The second resilient member 82 is disposed between and resiliently abutting against the implant seat 70 and the needle extraction member 90. The second resilient member 82 is a compression spring.

The needle implant member 100 includes a needle implant member body 110, and a needle 120 connected to the needle implant member body 110 for carrying a sensor 502.

Referring to FIG. 5, the fixture members 3 are respectively and slidably disposed in the slide grooves 515 of the main body member 51. Each of the fixture members 3 has an abutment portion 301, a support portion 302 opposite to the abutment portion 301, a first hook 303 disposed between the abutment portion 301 and the support portion 302, and a driver portion 304 disposed between the abutment portion 301 and the support portion 302 and having a driver surface 305 that is operable to be pushed by a respective one of the push members 35. When the bottom cover 40 is coupled to the outer shell 10, the bottom cover 40 blocks the fixture members 3, which positions the fixture members 3 relative to the main body member 51 (i.e., the fixture members 3 are not movable relative to the main body member 51).

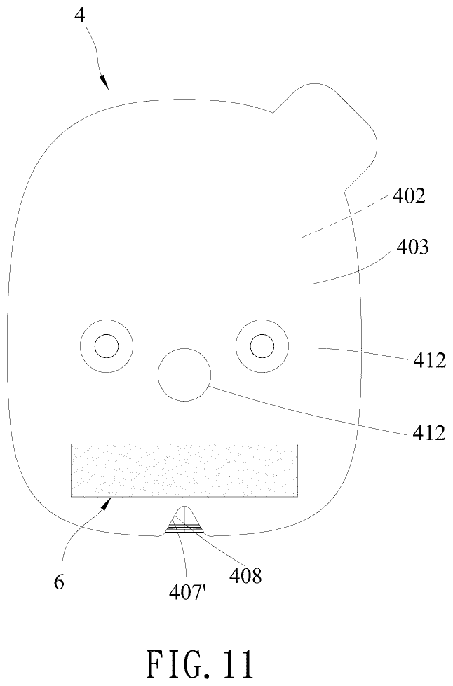

Referring to FIGS. 2, 11 and 12, the patch base 4 is removable from and positioned relative to the main body unit 50. The patch base 4 includes a base body 401, a bonding layer 420, an adhesive pad 402 connected fixedly to the base body 401, and an outer release layer 403. The bonding layer 420 is disposed between the base body 401 and the adhesive pad 402. The base body 401 has a sensor positioning groove 400, a periphery 404, and two engagement grooves 405 recessing into a bottom side of the periphery 404. The periphery 404 has two first sides 406, and two second sides 407 interconnecting the first sides 406. A length of each of the first sides 406 is equal to or shorter than a length of each of the second sides 407. In this embodiment, the outer release layer 403 is attached detachably to the adhesive pad 402. The base body 401 of the patch base 4 is made of a material harder than a material of the adhesive pad 402. The first hooks 303 of the fixture members 3 are respectively and removably engaged with the engagement grooves 405. Referring to FIGS. 11 and 12, the adhesive pad 402 has a first notch 407′ corresponding in position to one of the first sides 406. The outer release layer 403 has a second notch 408 corresponding in position to the first notch 407′. A material of the base body 401 may be polycarbonate (PC), acrylonitrile butadiene styrene (ABS), a blend of polycarbonate/acrylonitrile butadiene styrene, or polypropylene (PP). According to ASTM D2240 or ISO 868 standard, the Shore durometers of the abovementioned materials all range from 50 to 90. In this embodiment, the Shore durometer of the base body 401 ranges from 70 to 80.

The sensor assembly 5 is removable from and positioned relative to the patch base 4. The sensor assembly 5 includes a sensor seat 501 and the sensor 502. The sensor 502 is connected to the sensor seat 501 and is disposed in the needle 120. The sensor seat 501 has two securing portions 503 that are respectively engaged with the link portions 781 and that are recesses (Due to the viewing angle causing components to overlap, only one securing portion 503 and one link portion 781 are shown in FIG. 3.). The support portions 302 cooperatively support the sensor seat 501 to position the sensor seat 501 relative to the main body member 51. In another embodiment, the securing portions 503 may be protrusions, the link portions 781 may be recesses that are respectively coupled to the securing portions 503 for preventing the sensor seat 501 from rotating before the sensor 502 is inserted into the user's skin.

The peel-off component 6 is connected to the patch base 4 and the bottom cover 40. Referring to FIGS. 11 to 13, the peel-off component 6 is connected to the bottom plate portion 41, and is disposed at an inner side of and is adjacent to the second notch 408 (i.e., the peel-off component 6 is disposed at an inner side of the one of the first sides 406). Specifically, the peel-off component 6 is connected to the outer release layer 403. The peel-off component 6 may be a type of foam tape, or another material with a specific thickness and resilience, and is adhereable on two sides. The peel-off component 6 may be a biocompatible material. In this embodiment, the peel-off component 6 is a foam tape, and the outer release layer 403 has a plurality of small holes 412. When the bottom cover 40 is separated from the outer shell 10, via the connection between the peel-off component 6 and the outer release layer 403, the peel-off component 6 removes the outer release layer 403 from the adhesive pad 402.

Referring to FIG. 12, the patch base 4 further includes an inner release layer 403′. The inner release layer 403′ is attached to the bonding layer 420. The outer release layer 403 is attached detachably to the adhesive pad 402. Referring further to FIG. 11, the base body 401, the bonding layer 420, the inner release layer 403′, the adhesive pad 402 and the outer release layer 403 are assembled through a first attaching step, a pre-hot pressing step, a second attaching step, and a hot pressing step, details of which are as follows.
(1) In the first attaching step, the bonding layer 420 is attached to the patch base 4; specifically, the inner release layer 403′ is attached to the bonding layer 420, and the bonding layer 420 is then attached to the base body 401. The bonding layer 420 is made of a polymeric material such as thermoplastic polyurethane (TPU), or ethylene vinyl acetate (EVA), and may be biocompatible.
(2) In the pre-hot pressing step, the bonding layer 420 is processed. The pre-hot pressing step is performed from the side of the inner release layer 403′ opposite to the bonding layer 420 and is performed in a direction toward the base body 401 to secure the bonding layer 420 on a bottom surface of the base body 401. The bonding layer 420 has at least one pre-hot pressing position 420′. Referring to FIG. 12, in this embodiment, the bonding layer 420 has four pre-hot pressing portions 420′. The pre-hot pressing step is performed for 3 seconds to 10 seconds at a temperature of 75° C. to 85° C. and under a pressure of 3.5 kg/cm² to 4.5 kg/cm². After the pre-hot pressing step, the inner release layer 403′ is removed from the bonding layer 420 before the second attaching step is performed.
(3) In the second attaching step, the adhesive pad 402 is attached to the bonding layer 420; specifically, the outer release layer 403 is attached to the adhesive pad 402, and the adhesive pad 402 is then attached to the bonding layer 420. The adhesive pad 402 has an adhesive surface 402′ attached to the outer release layer 403, and at least one hot pressing position 402″. In this embodiment, the adhesive pad 402 has four hot pressing positions 402″.
(4) In the hot pressing step, the adhesive pad 402 is processed. The hot pressing step is performed from a side of the outer release layer 403 opposite to the adhesive pad 402 and is performed in a direction toward the base body 401 to secure the adhesive pad 402 on the base body 401 via the bonding layer 420. The hot pressing step is performed for 10 seconds to 20 seconds at a temperature of 115° C. to 125° C. and under a pressure of 3.5 kg/cm² to 4.5 kg/cm².

An adhering step of the peel-off component 6 is as follows. The peel-off component 6 is disposed between the bottom cover 40 and the outer release layer 403, and corresponds in position to one of the hot pressing positions 402″ of the adhesive pad 402 and the pre-hot pressing positions 420′ of the bonding layer 420.

To provide insight into effects caused by the cooperation of the components in the disclosure, technical means used, and expected results, further elaboration is as follows, which may help the reader have deeper and concrete understanding of the disclosure.

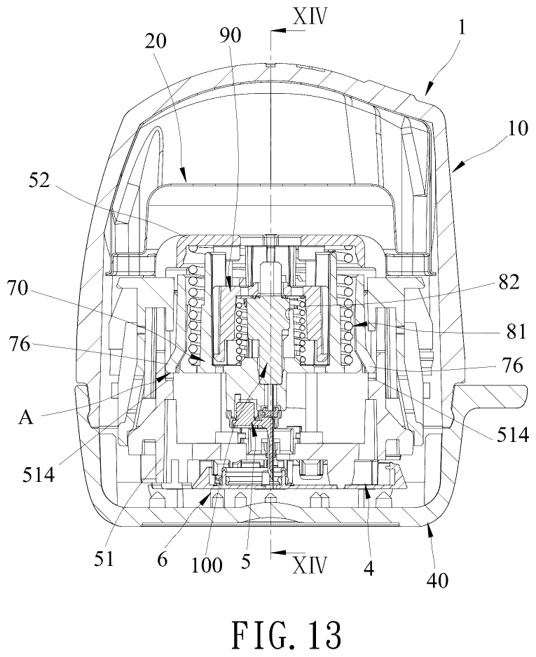

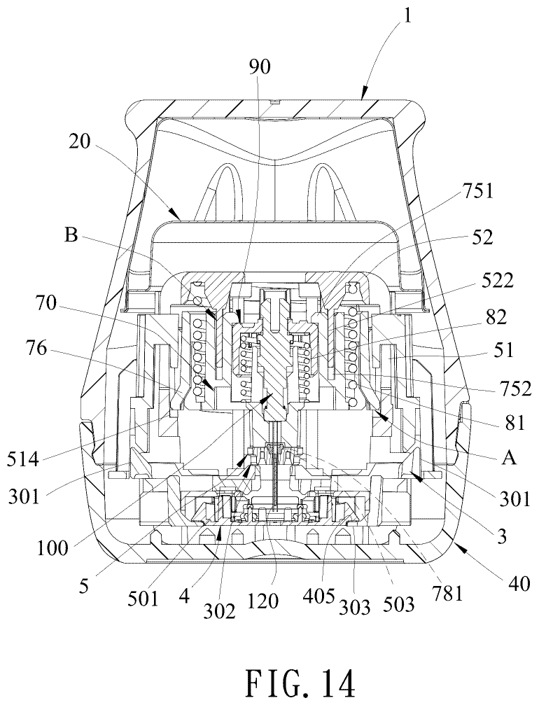

Referring to FIGS. 1 to 3, 13 and 14, when the insertion device is assembled, the bottom cover 40 is airtightly coupled to the outer shell 10. At this time, the main body member 51 and the main body cap 52 are integrated with each other via engagement between the engagement portions 513 and the connection portions 521 (see FIG. 3). When the insertion device is in an unfired state, there is a distance between the top cap 20 of the cover assembly 1 and the main body cap 52, and the buckle portions 76 of the implant seat 70 respectively engage the buckle stopper portions 514 of the main body member 51 to keep the implant seat 70 at an upper position. The needle implantation limit structure (A) formed by the implant seat 70 and the main body member 51 produces a safety lock to lock a position of the implant seat 70 relative to the main body member 51. The first resilient member 81 is compressed and stores a resilient force. The limit members 522 are respectively slotted in the limiter slots 752, which limits movement of the limiter components 751 in a radial direction. The needle extraction limit structure (B) forms another safety lock that locks a position of the needle extraction member 90 relative to the implant seat 70. The second resilient member 82 is compressed and stores a resilient force. The needle extraction member 90 is at an unfired position (see FIGS. 13 and 14). The securing portions 503 of the sensor assembly 5 are respectively coupled to the link portions 781, so that the sensor seat 501 is connected to the implant seat 70. The needle 120 of the needle implant member 100 is disposed within the main body member 51, and a tip of the needle 120 does not protrude from the patch base 4. The patch base 4 is positioned relative to the main body member 51. At this time, the bottom cover 40 abuts against the abutment portions 301, which positions the fixture members 3 relative to the main body member 51. The support portions 302 cooperatively support the sensor seat 501 10 to position the sensor seat 501 relative to the main body member 51. The first hooks 303 respectively engage the engagement grooves 405.

When the sensor 502 is to be implanted under the user's skin, an implant process is performed as follows.

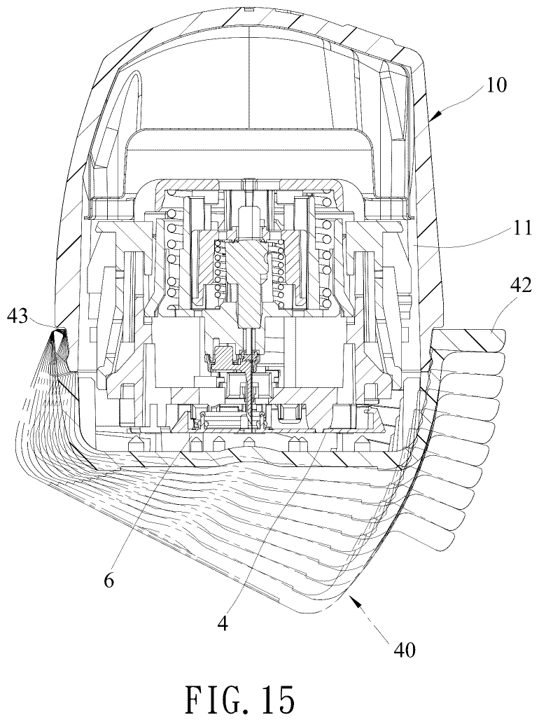

Referring to FIG. 15, the user first removes the bottom cover 40 from the outer shell 10. The bottom cover 40 has a force exertion portion 42 for receiving a force, and a pivot portion 43 formed opposite to the force exertion portion 42. When the force is exerted on the force exertion portion 42, a force-exertion torque is formed, with the pivot portion 43 and the force exertion portion 42 acting collectively as a single torque arm to rotate about the pivot portion 43. The length of the torque arm is defined by a distance between the force exertion portion 42 and the pivot portion 43. The force-exertion torque allows the user to form a partial side opening between the bottom cover 40 and the outer shell 10 via the force exertion portion 42. When the bottom cover 40 is being removed from the outer shell 10, the outer release layer 403 is detached from the adhesive pad 402 via the connection between the peel-off component 6, the bottom plate portion 41 of the bottom cover 40, and the outer release layer 403. After the outer release layer 403 is detached from the adhesive pad 402, the user can adhere the adhesive pad 402 of the bottom cover 40 to a desired body part (not shown) where the sensor 502 is to be implanted.

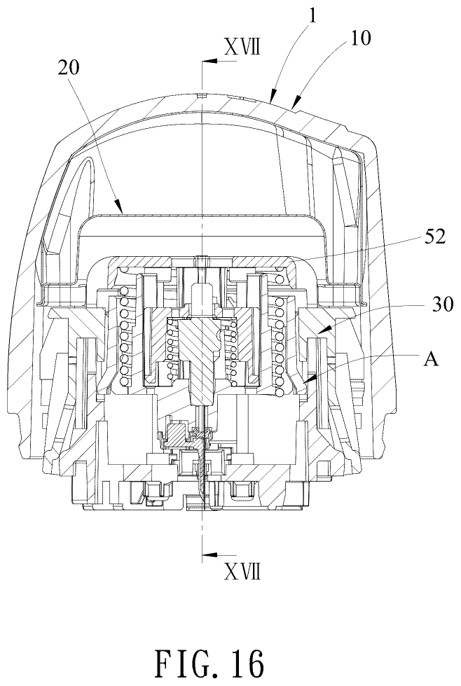

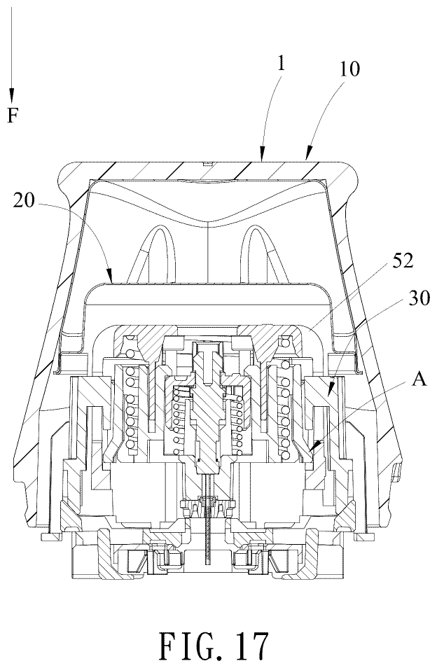

Referring to FIGS. 16 and 17, when the user pushes the outer shell 10 of the cover assembly 1 in a needle implantation direction (F), the outer shell 10 drives the top cap 20 and the inner lining member 30 to move towards the desired body part. Specifically, the inner lining member 30 is moved to a lower critical position in preparation for unlocking the needle implantation limit structure (A). At this time, the main body cap 52 is not displaced.

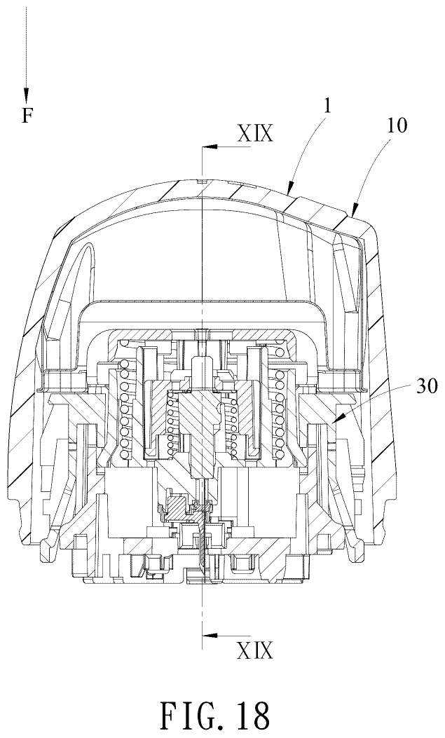

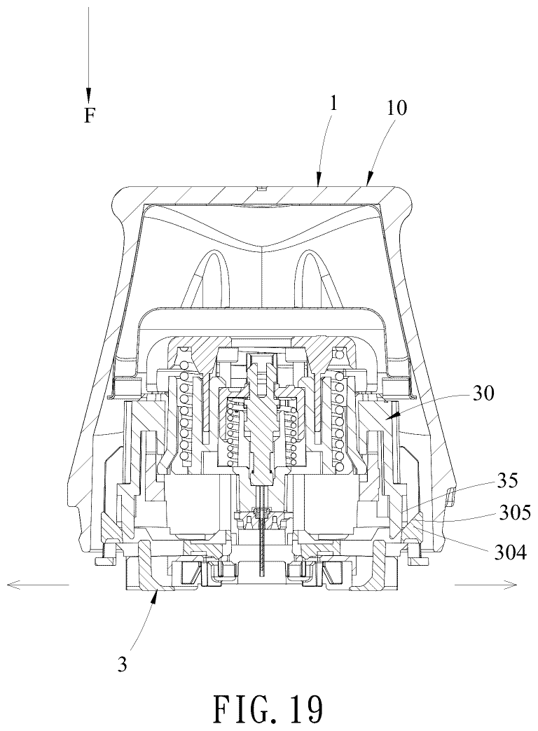

Referring to FIGS. 18 and 19, when the user further pushes the outer shell 10, the push members 35 of the inner lining member 30 are driven to respectively push against the driver portions 304, thereby driving the fixture members 3 to respectively slide outwards along the slide grooves 515. The abovementioned movement of the components form an unlocking step of the fixture members 3 before the needle 120 is fired, which removes restrictions on the relative positions of the sensor assembly 5 and the patch base 4.

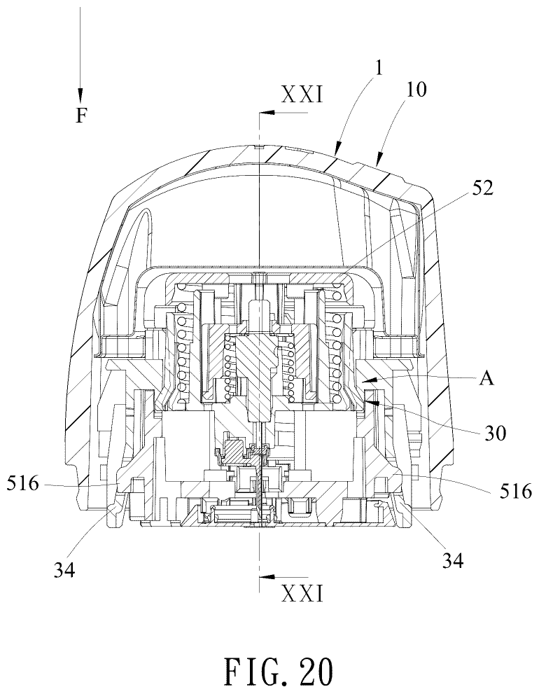

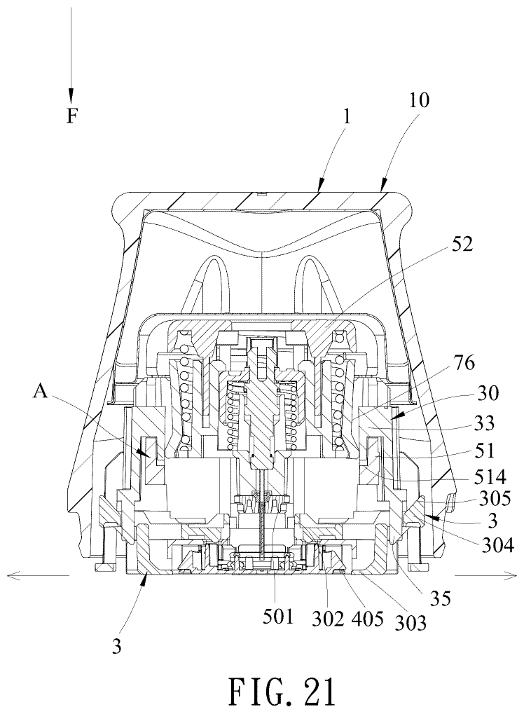

Referring to FIGS. 20 and 21, the outer shell 10 is further pushed so that a distance between the inner lining member 30 and the main body cap 52 is shortened or closed. At this time, the firing portions 33 of the inner lining member 30 respectively drive the buckle portions 76 of the implant seat 70 to move to be respectively disengaged from the buckle stopper portions 514 (see FIG. 21). Meanwhile, the latch portions 34 respectively engage the lock portions 516 (see FIG. 20), so that the inner lining member 30 is secured on the main body member 51. The needle implantation limit structure (A) is destroyed when the buckle portions 76 of the implant seat 70 are disengaged from the buckle stopper portions 514 of the main body member 51, which removes the restriction on the relative positions of the main body member 51 and the implant seat 70. When the cover assembly 1 is pushed, the push members 35 of the inner lining member 30 respectively push against the driver portions 304 of the fixture members 3, thereby driving the fixture members 3 to simultaneously slide outwards relative to the main body member 51, so that the sensor seat 501 loses the support of the support portions 302, and that the first hooks 303 are disengaged from the respective engagement grooves 405. . . . The driver surfaces 305 allow smooth movement of the push members 35 in driving the fixture members 3 to move.

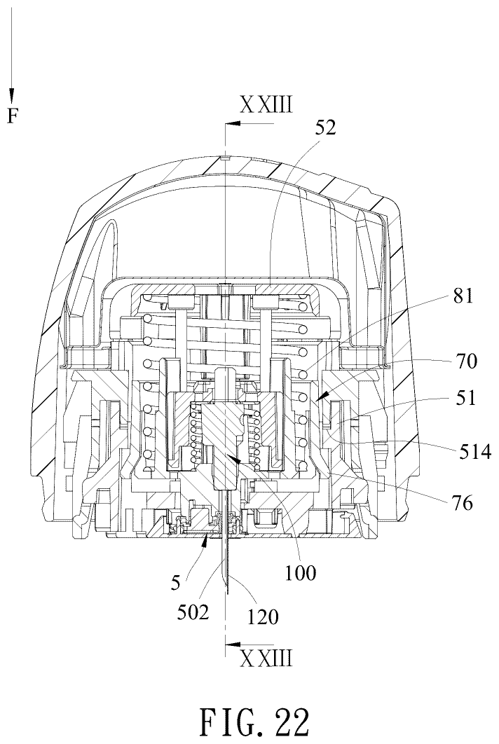

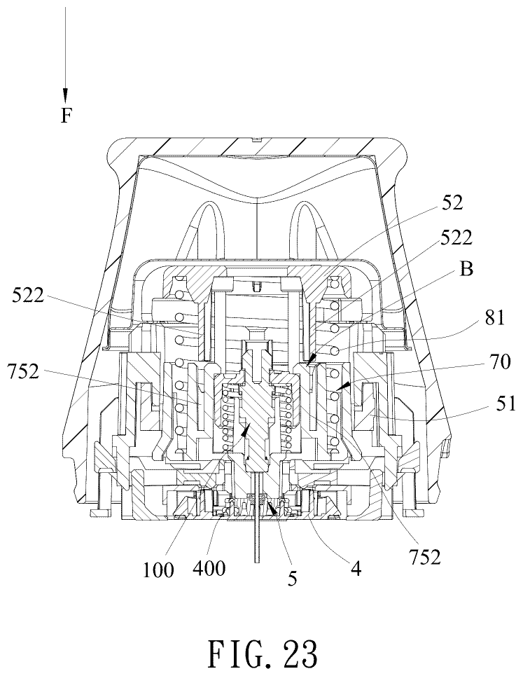

Referring to FIGS. 22 and 23, when the buckle portions 76 of the implant seat 70 are disengaged from the buckle stopper portions 514 of the main body member 51, the restriction on the relative positions of the main body member 51 and the implant seat 70 is removed so that the resilient force stored in the first resilient member 81 can be released. The resilient force stored in the first resilient member 81 biases the implant seat 70 to move away from the main body cap 52 in the needle implantation direction (F). Specifically, the implant seat 70 is biased to a lower position, the sensor assembly 5 is driven by the implant seat 70 and the needle implant member 100 to move to a fired position, where the needle 120 penetrates the skin, the sensor 502 that is carried in the needle 120 is implanted under the skin, and the sensor seat 501 of the sensor assembly 5 is engaged with the sensor positioning groove 400 of the patch base 4. At this time, the limiter members 522 of the main body cap 52 are disengaged from the limiter slots 752.

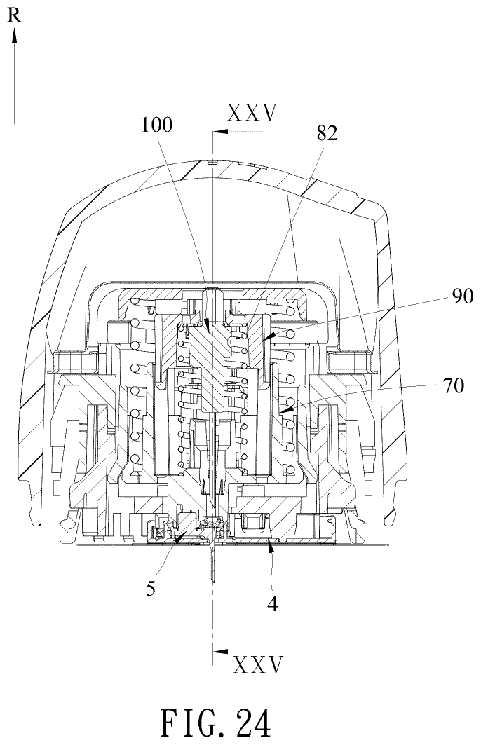

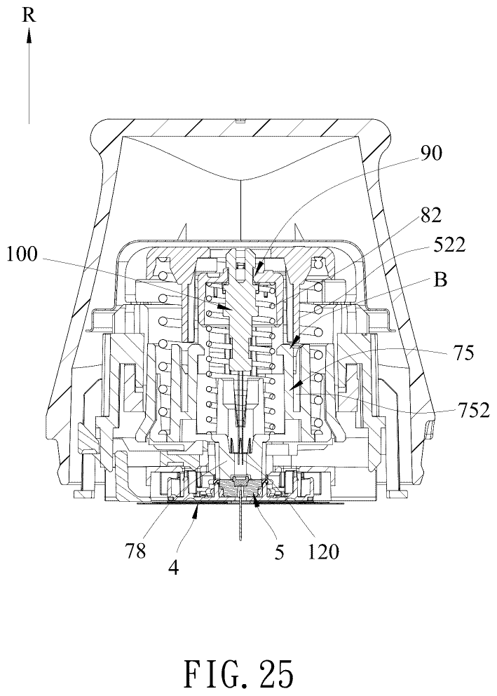

Afterwards, referring to FIGS. 24 and 25, since the limiter members 522 of the main body cap 52 are disengaged from the limiter slots 752, movement of the limiter components 751 in the radial direction is no longer restricted, thereby unlocking the positional restriction the limiter module 75 has on the needle extraction member 90. The resilient force stored inside the second resilient member 82 is released, which biases the needle extraction member 90 to drive the needle implant member 100 to move in a needle extraction direction (R) opposite to the needle implantation direction (F), so as to extract the needle 120 from the skin and into the implant seat 70. The positioner member 78 keeps pressing the sensor assembly 5 to the patch base 4, which prevents the kinetic energy resulting from the needle 120 being extracted from causing the sensor assembly 5 to bounce back.

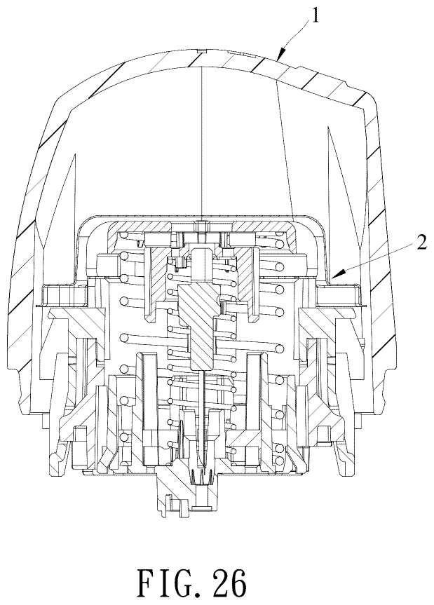

Referring to FIG. 26, when the user separates and lifts the cover assembly 1 and the implant module 2 away from the skin and the patch base 4, the patch base 4 and the sensor assembly 5 remain attached to the skin (only the cover assembly 1 and the implant module 2 are shown in FIG. 26, the patch base 4 and the sensor assembly 5 that are attached to the skin are not shown).

Afterwards, the user reattaches the bottom cover 40 that was removed to the bottom of the outer shell 10, and seals the bottom of the outer shell 10 with the bottom cover 40.

Effects of the disclosure are as follows.
1. Referring to FIGS. 11 and 12, the peel-off component 6 is disposed at an inner side of the periphery 404 of the base body 401. The base body 401 is rigid, so that the base body 401 can serve as a supporting fixture during removal of the outer release layer 403, which causes forces to concentrate around the peel-off component 6 and be utilized more effectively allowing for concentrated peeling torque; accordingly, the success rate of removing the outer release layer 403 via the peel-off component 6 is at 99%. If the peel-off component 6 is provided on, for example, a soft textile component, the success rate of removing the outer release layer 403 via the peel-off component 6 will be greatly lowered.
2. The adhesive pad 402 has the first notch 407′, and the outer release layer 403 has the second notch 408. The first notch 407′ and the second notch 408 allow air to escape, which decreases resistance in the removal process of the outer release layer 403, and makes the removal process easier. Adding a notch or a gap at a periphery and/or a set location of the outer release layer 403 creates a starting point for the removal process of the outer release layer 403, making the removal process easier.
3. The outer release layer 403 has microstructures, for example the small holes 412 or small bumps, which decrease adhesion. Furthermore, holes or slots can weaken the structure of the outer release layer 403. When designing a removal path of the outer release layer 403, the inventor may consider the structural weakness thereof, so as to make the removal process easier.
4. The outer release layer 403 is attached to the adhesive pad 402 that has been hot-pressed. After the adhesive pad 402 has been hot-pressed, the portions of the adhesive pad 402 at the hot pressing positions 402″ have adhesion less than that of other portions of the adhesive pad 402 that were not hot-pressed, which is advantageous for removal of the outer release layer 403 from the adhesive pad 402 via the peel-off component 6 when the bottom cover 40 is being removed from the outer shell 10.

In conclusion, when the bottom cover 40 is removed from the outer shell 10, the outer release layer 403 can be simultaneously removed from the adhesive pad 402, so that the insertion device can be quickly secured onto the user's skin, thereby achieving convenient and quick usage. Hence, the objective of the disclosure is achieved.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An insertion device adapted to be installed in a biosensor, said insertion device comprising:
a cover assembly including an outer shell and a bottom cover that is removably coupled to said outer shell, said outer shell defining an accommodating space;
an implant module disposed in said accommodating space and operable for implanting and extracting a needle;
a patch base positioned removably relative to said implant module, and including
a base body,
an adhesive pad that is connected fixedly to said base body, and
an outer release layer that is attached detachably to said adhesive pad;
a sensor assembly connected to said base body when said needle is implanted by said implant module; and
a peel-off component connected to said bottom cover and said outer release layer;
wherein said peel-off component removes said outer release layer from said adhesive pad when said bottom cover is separated from said outer shell.

2. The insertion device as claimed in claim 1, wherein:
said base body of said patch base is made of a material harder than a material of said adhesive pad;
said base body has a periphery; and
said peel-off component is disposed at an inner side of said periphery.

3. The insertion device as claimed in claim 2, wherein:
said periphery of said base body has two first sides, and two second sides interconnecting said first sides;
said adhesive pad has a first notch corresponding in position to one of said first sides;
said outer release layer has a second notch corresponding in position to said first notch; and
said peel-off component is disposed at an inner side of and is adjacent to said second notch.

4. The insertion device as claimed in claim 1, wherein:
said patch base further includes a bonding layer disposed between said base body and said adhesive pad;
said bonding layer is pre-hot-pressed and secured to said base body; and
said adhesive pad is hot-pressed and connected to said base body via said bonding layer.

5. The insertion device as claimed in claim 4, wherein:
said patch base further includes an inner release layer attached to said bonding layer; and
said inner release layer is pre-hot-pressed from a side thereof opposite to said bonding layer to secure said bonding layer on said base body.

6. The insertion device as claimed in claim 5, wherein:
said base body, said bonding layer, said inner release layer, said adhesive pad and said outer release layer are assembled through a first attaching step, a pre-hot pressing step, a second attaching step, and a hot pressing step;
in the first attaching step, said inner release layer is attached to said bonding layer, and said bonding layer is then attached to said base body;
the pre-hot pressing step is performed from said side of said inner release layer opposite to said bonding layer and is performed in a direction toward said base body to secure said bonding layer on said base body;
after the pre-hot pressing step, said inner release layer is removed from said bonding layer before the second attaching step is performed;
in the second attaching step, the outer release layer is attached to said adhesive pad, and said adhesive pad is then attached to said bonding layer; and
the hot pressing step is performed from a side of said outer release layer opposite to said adhesive pad and is performed in a direction toward said base body to secure said adhesive pad on said base body via said bonding layer.

7. The insertion device as claimed in claim 6, wherein:
said bonding layer is made of a polymeric material, and has at least one pre-hot pressing position; and
said adhesive pad has at least one hot pressing position.

8. The insertion device as claimed in claim 7, wherein the pre-hot pressing step is performed for 3 seconds to 10 seconds at a temperature of 75° C. to 85° C. and under a pressure of 3.5 kg/cm² to 4.5 kg/cm².

9. The insertion device as claimed in claim 8, wherein the hot-pressing step is performed for 10 seconds to 20 seconds at a temperature of 115° C. to 125° C. and under a pressure of 3.5 kg/cm² to 4.5 kg/cm².

10. The insertion device as claimed in claim 7, wherein said peel-off component is disposed between said bottom cover and said outer release layer, and corresponds in position to at least one of said at least one hot pressing position of said adhesive pad and said at least one pre-hot pressing position of said bonding layer.
